(19) United States
(12) Patent Application Publication
(10) Pub. No.: US 2008/0281073 A1
(43) Pub. Date: Nov. 13, 2008

(54) High-Purity Oxydiphthalic Acid Anhydride and Process for Producing the Same

(75) Inventors: Kazuhiro Nagayama, Kanagawa (JP); Toshiharu Yokoyama, Kanagawa (JP); Jun Takahara, Kanagawa (JP); Makoto Nitta, Fukuoka (JP); Hiroshi Mikami, Fukuoka (JP)

Correspondence Address: OBLON, SPIVAK, MCCLELLAND MAIER & NEUSTADT, P.C., 1940 DUKE STREET, ALEXANDRIA, VA, 22314, US

(73) Assignee: MITSUBISHI CHEMICAL CORPORATION, Tokyo (JP)

(21) Appl. No.: 11/577,938

(22) PCT Filed: Dec. 7, 2005
(86) PCT No.: PCT/JP05/22487

§ 371 Date: Apr. 25, 2007

(30) Foreign Application Priority Data
Dec. 7, 2004 (JP) 2004-353696

(51) Int. Cl. C08G 69/36; B32B 5/16; C07D 307/60

(52) U.S. Cl. 528/329.1; 428/402; 549/250

(57) ABSTRACT

It is to provide a high purity oxydiphthalic anhydride with which, in production of a polyimide containing an oxydiphthalic anhydride, one having sufficiently high strength can be produced, and an industrially simple process for producing it.

A high purity oxydiphthalic anhydride, which has a content of fine insoluble particles having a projected area diameter of from 5 to 20 μm, of at most 3,000 particles per 1 g, and has a light transmittance at 400 nm of at least 98.5% in a light path length of 1 cm when dissolved in acetonitrile at a concentration of 4 g/L.

### TECHNICAL FIELD

The present invention relates to a high purity oxydiphthalic anhydride suitable as a monomer for a high definition photosensitive polyimide in a semiconductor production field, and its production process.

### BACKGROUND ART

An oxydiphthalic anhydride (hereinafter sometimes referred to as ODPA) is a monomer which imparts transparency and thermal plasticity to a polyimide having high heat resistance. Accordingly, ODPA is utilized as a raw material for a polyimide to be used for transparent polyimide films and electronic material- and semiconductor-related application.

As industrially advantageous processes for producing ODPA, a process of coupling nitrophthalic acid (anhydride) in the presence of nitrous acid (nitrite) (Patent Document 1), a process of reacting phthalimide which may have a substituent with a nitrite and/or a carbonate in a stoichiometric amount to convert the phthalimide to diaryl ether, hydrolyzing the imide ring and further converting the tetracarboxylic acid to an anhydride (Patent Document 2, and a process of reacting two molecules of a halogenated phthalic anhydride with a carbonate in an stoichiometric amount in the presence of a phase-transfer catalyst such as a phosphonium salt for coupling (Patent Document 3) have been known.

As methods of purifying such a crude ODPA thus obtained, a method of washing it with an organic solvent such as acetic acid (Patent Document 3) and a purification method by a process of hydrolyzing it in an aqueous propionic acid solution to a tetracarboxylic acid, followed by heating for dehydrative cyclization to obtain an acid dianhydride again (Patent Document 4) have been known.

Patent Document 1: JP-A-55-136246

Patent Document 2: Chinese Patent No. 1036065

Patent Document 3: Japanese Paten No. 3204641

Patent Document 4: JP-B-7-98774

### DISCLOSURE OF THE INVENTION

### Problems that the Invention is to Solve

The present inventors have produced a polyimide film employing a purified ODPA obtained by washing with an organic solvent a crude ODPA produced by the above-described process, and evaluated its quality. As a result, such a phenomenon has been quite often observed that the film have broken before the yield point in a tensile test. That is, even when a polyimide is produced employing an ODPA produced by a known process, the polyimide to be obtained has low strength and has no sufficient quality as a product.

It is an object of the present invention to provide an ODPA to be used to obtain a polyimide having sufficient strength in production of a polyimide from an ODPA and a diamine, and an industrially simple process for producing it.

### Means of Solving the Problems

The present inventors have conducted extensive studies to achieve the above object and as a result found that a polyimide produced by employing an ODPA purified by a specific process, has improved strength and transparency, and further found that a decrease in strength of the polyimide is attributable to specific impurities. The present invention has been accomplished on the basis of these discoveries.

Namely, the present invention provides the following:

(1) A high purity oxydiphthalic anhydride, which has a content of fine insoluble particles having a projected area diameter of from 5 to 20 μm of at most 3,000 particles per 1 g. and has a light transmittance at 400 nm of at least 98.5% in a light path length of 1 cm when dissolved in acetonitrile at 4 g/L.

(2) The high purity oxydiphthalic anhydride according to the above (1) which has a halogen atom content of at most 9 μmol/g.

(3) The high purity oxydiphthalic anhydride according to the above (1) or (2), which has a nitrogen atom content of at most 14 μmol/g.

(4) A process for producing a high purity oxydiphthalic anhydride, which comprises purifying a crude oxydiphthalic anhydride by a procedure comprising the following steps A and B:

step A: a step of heating the crude oxydiphthalic anhydride to a temperature of at least 150° C. and at most 350° C. to evaporate and/or sublimate it, and condensing and recovering the evaporated and/or sublimated vapor;

step B: a step of washing the crude oxydiphthalic anhydride with at least one solvent selected from an organic acid having at most 6 carbon atoms, and an organic ester or a ketone having at most 12 carbon atoms in an amount of from 3.5 to 20 times the weight of the crude oxydiphthalic anhydride.

(5) The process for producing a high purity oxydiphthalic anhydride according to the above (4), wherein a crude oxydiphthalic anhydride obtained by reacting a halogenated phthalic acid with a carbonate or a halogenated phthalate, is purified by a procedure of carrying out the step A and then the step B.

(6) The process for producing a high purity oxydiphthalic anhydride according to the above (4), wherein a crude oxydiphthalic anhydride obtained by coupling a substituted phthalimide, is purified by a procedure of carrying out the step B and then the step A.

(7) A process for producing a high purity oxydiphthalic anhydride, which comprises heating a crude oxydiphthalic anhydride having a nitrogen atom content of at most 14 μmol/g to a temperature of at least 150° C. and at most 350° C. to evaporate and/or sublimate it, and condensing and recovering the evaporated and/or sublimated vapor.

(8) The process for producing a high purity oxydiphthalic anhydride according to the above (7), wherein the crude oxydiphthalic anhydride is a crude oxydiphthalic anhydride obtained by coupling a substituted phthalimide.

(9) A high purity oxydiphthalic anhydride produced by the production process as defined in any one of the above (4) to (8).

(10) A polyimide containing the high purity oxydiphthalic anhydride as defined in any one of the above (1) to (3) and (9) as a component.

(11) A polyimide containing oxydiphthalic anhydride units and diamine units wherein a film of the polyimide having a thickness of 20 μm a length of 50 mm (a length at a stretched portion of 20 mm) and a width of 10 mm, has a breaking extension of at least 25% as measured in accordance with JIS K7113.

(12) The polyimide containing oxydiphthalic anhydride units and diamine units according to the above (11), wherein a film of the polyimide having a thickness of 20 μm, a length of 50 mm (a length at a stretched portion of 20 mm) and a width of 10 mm, has a breaking stress of at least 130 MPa as measured in accordance with JIS K7113.

### EFFECTS OF THE INVENTION

The high purity ODPA of the present invention is to provide a high quality ODPA suitable particularly for production of a polyimide, and by polymerizing it with a diamine, a high viscous polyamic acid can be produced. Further, a highly heat resistant and highly transparent polyimide film, and a high definition photosensitive polyimide useful in a semiconductor production field, which have sufficiently high strength, can be produced with a very low percent defective.

Further, according to the process for producing a high purity ODPA of the present invention, a high purity ODPA can be produced by an industrially advantageous and simple procedure.

### BEST MODE FOR CARRYING OUT THE INVENTION

### 1. Process for Producing High Purity ODPA

### (1) Crude ODPA

The process for producing a crude ODPA to be purified is not particularly limited, and any ODPA produced by a known process such as a process as disclosed in JP-A-55-136246, Chinese Patent No. 1036065 or Japanese Patent No. 3204641 may be used. Typically, ODPA produced by the following processes (1-1) to (1-3) are preferred.

### (1-1) Production Process Using Nitrophthalic Acid or Nitrophthalic Anhydride as Starting Raw Material

This process is to convert nitrophthalic acid or its anhydride to a diaryl ether in the presence of nitrous acid or a nitrite to produce oxydiphthalic acid or ODPA. This process will be described in detail below.

(a) Nitrophthalic Acid or its Anhydride

In this process, either nitrophthalic acid or nitrophthalic anhydride is used. However, in the case of nitrophthalic acid, a step of converting oxydiphthalic acid to be obtained after coupling to the acid anhydride is further required. Accordingly, it is preferred to use nitrophthalic anhydride which can be directly converted to ODPA as a substrate. The nitrophthalic anhydride is preferably one represented by the following formula (1). The substitution position of the nitro group on the aromatic ring is not particularly limited, and either 3-form or 4-form may be used. Such isomers may be used alone or may be used as a mixture for the reaction.

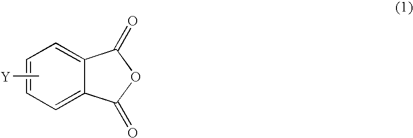

wherein Y is a nitro group.

(b) Nitrous Acid or Nitrite

In this reaction nitrous acid or a nitrite is used as a reaction catalyst. A nitrite is particularly preferred. The nitrite to be used is usually a nitrite of an alkali metal or an alkaline earth metal, and among them, sodium nitrite is preferably used.

The amount of the nitrous acid or the nitrite to be used for the reaction is not particularly limited relative to nitrophthalic acid or its anhydride as the reaction substrate, and it is used usually in an amount of at most 1 by the ratio of the amount of substance, preferably the addition amount is from 0.05 to 20 mol % as a nitrous acid group.

(c) Reaction Solvent

In this process, the reaction is carried out in an aprotic polar solvent, and the solvent is not particularly limited. Usually, dimethyl sulfoxide, sulfolane, N,N-dimethylformamide, N,N-dimethylacetamide, N-methylpyrrolidone, hexamethylphosphoric triamide, etc. may be preferably used. As the amount of the solvent to be used, it is used in such a range that the upper limit of the concentration of the nitrophthalic acid or the nitrophthalic anhydride is usually at least 1 wt %, preferably at least 5 wt %, and the upper limit is usually at most 30 wt %, preferably at most 20 wt %.

(d) Reaction Method

As the reaction temperature, the lower limit is usually at least 50° C., preferably at least 80°, and the upper limit is usually at most 200° C., preferably at most 150° C. The reaction is carried out usually under atmospheric pressure, but may be carried out under reduced pressure or under elevated pressure.

The reaction may be carried out in the air. However, it is carried out preferably in an inert gas atmosphere of e.g. nitrogen or argon. The reaction time is preferably at least 0.5 hour and at most 24 hours. After completion of the reaction, usually in accordance with a known method, the solvent is removed under reduced pressure and deposited solid is washed with water, whereby an aimed crude ODPA or crude oxydiphthalic acid will be obtained. The oxydiphthalic acid is converted to ODPA in accordance with a known method such as reaction with acetic anhydride or heating together with an organic solvent to 100° or higher for dehydration.

(1-2) Production Process Using Phthalimide which May have a Substituent as Starting Raw Material

This process is to react a phthalimide which may have a substituent with nitrous acid or a nitrate, and as the case requires, further with a carbonate to convert the phthalimide to a diaryl ether, hydrolyzing the imide ring and further converting the tetracarboxylic acid to an anhydride. This process will be described in detail below.

(a) Phthalimide which May have a Substituent

The phthalimide to be used in this process is preferably one represented by the following formula (2). The substitution position of the nitro group on the aromatic ring is not particularly limited, and either 3-form or 4-form may be used. R is usually one member selected from a hydrogen atom, a methyl group and an ethyl group, particularly preferably a methyl group. Such isomers may be used alone or may be used as a mixture for the reaction.

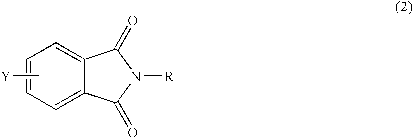

wherein Y is a nitro group, and R is a hydrogen atom or a hydrocarbon group.

The number of carbon atoms in the hydrocarbon group as R is usually at least 1, and usually at most 6, preferably at most 4, more preferably at most 3, furthermore preferably at most 2. The hydrocarbon group is preferably an alkyl group, an alkenyl group, an alkynyl group or an arylene group, and preferably an alkyl group. Among alkyl groups, a methyl group, an ethyl group or a propyl group is preferably mentioned, and a methyl group or an ethyl group is more preferred.

Among them, a hydrogen atom, a methyl group or an ethyl group is preferred.

(b) Nitrous Acid or Nitrite

The nitrous acid or the nitrite to be used in this reaction is preferably a nitrite. The nitrite is usually a nitrite of an alkali metal or an alkaline earth metal. The nitrite of an alkali metal or an alkaline earth metal is preferably sodium nitrite.

The amount of the nitrous acid or the nitrite to be used for the reaction is not particularly limited. It is used usually in an amount of at most 1 by the ratio of the amount of substance based on the nitrophthalimide as a reaction substrate, preferably in an amount of from 0.05 to 20 mol % as a nitrous acid group.

(c) Carbonate

In the reaction to a diaryl ether, the reaction activity will improve when a carbonate is added as a second catalytic component in addition to the nitrite. The carbonate to be used is lithium carbonate, sodium carbonate, potassium carbonate, rubidium carbonate, magnesium carbonate or calcium carbonate. From the viewpoint of reactivity and availability, preferred is potassium carbonate, sodium carbonate, lithium carbonate or cesium carbonate, and most preferred is potassium carbonate.

As the amount of the carbonate to be used, it is used in an amount of usually from 10 to 40 mol %, preferably from 20 to 35 mol %, as the ratio of the amount of substance based on the nitrite.

(d) Reaction Solvent

In this process, the solvent is not particularly limited, and the reaction is carried out preferably in an aprotic polar solvent. Usually, dimethyl sulfoxide, sulfolane, N,N-dimethylformamide, N,N-dimethylacetamide, N-methylpyrrolidone, hexamethylphosphoric triamide, etc. may be preferably used. It is also possible to use a solvent mixture comprising such a solvent and an aprotic solvent such as toluene or xylene added, for the purpose of controlling the reaction temperature. As the amount of the solvent to be used, it is used in such a range that the lower limit of the concentration of the nitrophthalimide is usually at least 1 wt %, preferably at least 5 wt %, and the upper limit is usually at most 30 wt %, preferably at most 20 wt %.

(e) Reaction Method

As the reaction temperature, the lower limit is usually at least 120° C., preferably at least 150° C., and the upper limit is usually at most 220° C., preferably at most 200° C. Particularly, a temperature of from 162 to 168° C. is most preferred. It is preferred to mix a plural types of reaction solvents so that the reflux temperature is adjusted to be within this temperature range. For example, the reflux temperature will be within this temperature range by mixing 1 mol of N-methyl-4-nitrophthalimide with 500 ml of N,N-dimethylacetamide and 150 ml of xylene. The reaction is carried out usually under atmospheric pressure, but may be carried out under reduced pressure or under elevated pressure.

The reaction may be carried out in the air. However, it is carried out preferably in an inert gas atmosphere of e.g. nitrogen or argon. The reaction time is preferably at least 0.5 hour, more preferably at least 1 hour, furthermore preferably at most 4 hours, and preferably at most 24 hours, more preferably at most 12 hours, furthermore preferably at most 8 hours. The reaction is initiated usually by heating the reaction raw materials to a predetermined reaction temperature with properly stirring.

(f) Treatment after Reaction

After completion of the reaction, in accordance with a known method, the solvent is removed by evaporation, deposited solid is washed with water, and the recovered solid is dried under reduced pressure usually at a temperature of 100° C. or higher to produce a diaryl ether bisimide represented by the following formula (3):

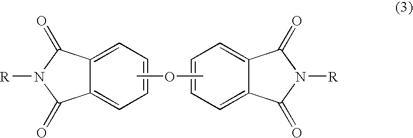

wherein R corresponds to R in the formula (2).

(g) Hydrolysis of Imide Ring

The diaryl ether bisimide is subsequently hydrolyzed by a known method and converted to an oxydiphthalic acid. This hydrolysis step is carried out usually by reacting a base in an aqueous solution. The amount of water to be used is usually from 1 to 100 times the weight of the diaryl ether bisimide. The base to be used is not particularly limited, and usually a hydroxide, a carbonate, a bicarbonate, a phosphate, a hydrogen phosphate, or an alkali metal, alkaline earth metal or ammonium salt of an organic carboxylic acid is usually used. A hydroxide or a carbonate is preferred, and sodium hydroxide is most preferred from an economical viewpoint. As the amount of the base, the lower limit is usually at least 1 equivalent, preferably at least 1.5 equivalents, more preferably at least 2 equivalents based on the diaryl ether bisimide, and the upper limit is usually at most 100 equivalents although it is not particularly limited. The reaction may be carried out at room temperature, but it is carried out usually with heating to from 70 to 100° C. so as to improve the reaction efficiency. The reaction is carried out under atmospheric pressure, but may be carried out under elevated pressure. The reaction time is usually from 0.5 to 24 hours. After completion of the reaction, it is possible to carry out a treatment by contact with activated carbon for the purpose of decoloring. The reaction liquid after filtration is cooled to room temperature, and subjected to an acid treatment, whereupon oxydiphthalic acid is deposited as white solid, which is subjected to filtration and dried to obtain oxydiphthalic acid. The acid to be added at the time of the acid treatment is not limited so long as it can neutralize the tetracarboxylate of the oxydiphthalic acids but usually hydrochloric acid, nitric acid or sulfuric acid is used. The amount of the acid to be added is at least the equivalent based on the amount of substance of the base used in the hydrolysis step, preferably within such a range that the pH of the solution after addition of the acid will be from 3 to 4.

(h) Conversion of Oxydiphthalic Acid to Anhydride

The oxydiphthalic acid is converted to an anhydride by a known method and thereby converted to the ODPA. For example, a method of reacting an acid anhydride with the oxydiphthalic acid, a method of refluxing the oxydiphthalic acid in an organic solvent such as o-dichlorobenzene with heating to remove water produced by intramolecular dehydration reaction by azeotropy, or a method of heating the oxydiphthalic acid in the form of solid to a temperature of at least 180° C., preferably at least 200° C. for dehydration, may be mentioned. Among them, a method of reacting an acid anhydride is preferred in view of a high rate of reaction. In this case, the acid anhydride to be used is not particularly limited, but acetic anhydride is preferred from the viewpoint of availability and economical efficiency. As the amount of the acid anhydride, it is used usually in an amount of at least 2 equivalents based on the amount of substance of the oxydiphthalic acid. In a case where the acid anhydride is a liquid, it may serve as a solvent, or it is possible to use an organic solvent, preferably an aromatic compound such as toluene or xylene as a solvent. The reaction may be carried out at room temperature, but is carried out usually at a temperature of at least 50° C. Although it is possible to carry out the reaction in the air, the reaction is carried out preferably in an inert gas atmosphere of e.g. nitrogen or argon. The reaction time is preferably at least 0.5 hour and at most 24 hours. After the reaction the solvent and the acid anhydride are vaporized by evaporation and removed, followed by drying to obtain the ODPA.

### (1-3) Process Using Halogenated Phthalic Anhydride as Starting Raw Material

This process is a process of reacting a halogenated phthalic anhydride i.e. phthalic anhydride whose hydrogen atom on the aromatic ring is substituted by a halogen atom, with a carbonate and/or a halogenated phthalate. This process will be explained in detail below.

(a) Halogenated Phthalic Anhydride

A halogenated phthalic anhydride represented by the following formula (4) is used:

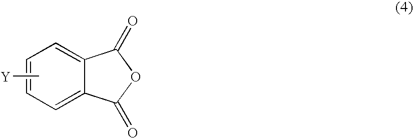

wherein Y is a halogen atom.

The halogen atom may be fluorine, chlorine, bromine or iodine, and fluorine, bromine or iodine is preferred A plural types of Y may be used in combination. Y is preferably chlorine or bromine in view of sufficiently high reactivity and easiness of production.

(b) Halogenated Phthalate

A halogenated phthalate represented by the following formula (5) is used:

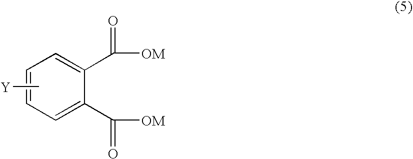

wherein Y is a halogen atom, and M is a hydrogen atom, or an alkali metal or alkaline earth metal atom.

The halogen atom as Y may be fluorine, chlorine, bromine or iodine, and chlorine, bromine or iodine is preferred. A plural types of Y may be used in combination. Among them, chlorine or bromine is preferred in view of a sufficiently high reactivity and easiness of production.

The alkali metal as M may be preferably lithium, sodium, potassium rubidium or cesium, and the alkaline earth metal may be preferably magnesium or calcium. A plural types of them may be used in combination. Among them, potassium or sodium is preferred in view of reactivity and availability.

The halogenated phthalate usually has moisture absorption characteristics, and a very small amount of water contained in it influences the reaction. Accordingly, it is required to be sufficiently dried preliminarily. The water content in the halogenated phthalate to be subjected to the reaction is preferably at most 0.2 wt %. As the halogenated phthalate is a solid at room temperature under normal pressure, it is required to be well pulverized so as to carry out the reaction efficiently. Preferably, it is used as a powder which passes through a sieving with a mesh size of 1 mm or smaller.

As the amount of the halogenated phthalate to be used in this reaction, the lower limit is usually at least 0.1 equivalent, preferably at least 0.5 equivalent, more preferably at least 0.8 equivalent by the ratio of the amount of substance (molar ratio) based on the halogenated phthalic anhydride, and the upper limit is usually at most 5 equivalents, preferably at most 2 equivalents, more preferably at most 1.2 equivalents.

(c) Carbonate

The carbonate to be used in this reaction is lithium carbonate, sodium carbonate, potassium carbonate, rubidium carbonate, magnesium carbonate or calcium carbonate, and from the viewpoint of the reactivity and availability, potassium carbonate, sodium carbonate or cesium carbonate is preferred.

As the amount of the carbonate to be used, the lower limit is usually at least 0.05 equivalent, preferably at least 0.25 equivalent, more preferably at least 0.4 equivalent by the ratio of the amount of mass (molar ratio) based on the halogenated phthalic anhydride, and the upper limit is usually at most 2.5 equivalents, preferably at most 1 equivalent, more preferably at most 0.6 equivalent.

(d) Catalyst

In this reaction, usually a catalyst is used. As the catalyst, a phosphonium salt, an ammonium salt, a guanidium salt or a sulfonium salt which is known as a phase-transfer catalyst is suitably used. As the onium salt, the phosphonium salt or the ammonium salt is represented by the formula (6)

R¹R²R³R⁴Q⁺X⁻ (6)

wherein Q is a nitrogen atom or a phosphorus atom, and the sulfonium salt is represented by the formula (7):

R⁵R⁶R⁷S⁺X⁻ (7)

In the formulae (6) and (7), each of R¹, R², R³, R⁴, R⁵, R⁶ and R⁷ which are independent of one another, is a hydrogen atom; an alkyl group such as a methyl group, an ethyl group or a propyl group; a cycloalkyl group such as a cyclohexyl group; an alkenyl group such as a vinyl group, a crotyl group or a phenylethenyl group; an alkynyl group such as an ethinyl group; an aryl group such as a phenyl group or a naphthyl group; or a heterocyclic group such as a pyridyl group or a furyl group. Each of R¹, R², R³, R⁴, R⁵, R⁶ and R⁷ has usually at most 20, preferably at most 10 carbon atoms. They may have a substituent, and such a substituent may, specifically, be an alkyl group such as a methyl group, an ethyl group or an octyl group, or an aryl group such as a phenyl group or a tolyl group.

R¹, R², R³, R⁴, R⁵, R⁶ and R⁷ may be the same or different, and one to three of them may be hydrogen atoms.

X is a halogen atom such as fluorine, chlorine, bromine or iodine, and among them, chlorine or bromine is preferred.

Among them, a phosphonium salt is preferred in view of thermal stability of the catalyst, and specifically, tetraphenylphosphonium bromide or tetraphenylphosphonium chloride is more preferred.

Further, it is possible to add an alkali metal halide as a second catalytic component. The alkali metal halide is preferably an iodide, most preferably potassium iodide.

As the amount of the catalyst to be used, the lower limit is usually at least 0.01%, preferably at least 0.1%, and the upper limit is usually at most 20%, preferably at most 15%, based on the weight of the substituted phthalic anhydride as a raw material.

(e) Reaction Solvent

This reaction may be carried out without a solvent. However, in order to decrease the viscosity of the reaction mixture and to carry out the reaction stably with a sufficient stirring efficiency, it is preferred to use a solvent. The solvent to be used has to be one which is essentially inert under reaction conditions and which has a sufficiently high boiling point. The boiling point of the solvent has to be at least 120° C., preferably at least 150° C. under normal pressure. The solvent which meets such requirements may, for example, be a chlorinated aromatic compound such as a dichlorobenzene, a trichlorobenzene or a dichlorotoluene, or benzonitrile, sulfolane, dimethyl sulfoxide, dimethylformamide, dimethylacetamide or N-methylpyrrolidone. The solvent is preferably a dichlorobenzene, a dichlorotoluene or a trichlorobenzene. As the amount of the solvent to be used, the lower limit is usually at least 10 wt %, preferably at least 20 wt %, and the upper limit is usually at most 500 wt %, preferably at most 200 wt %, based on the substituted phthalic anhydride.

(f) Reaction Method

As the reaction temperature, the lower limit is usually at least 150° C., preferably at least 180° C., and the upper limit is usually at most 260° C., preferably at most 250° C. The reaction is carried out usually under normal pressure, but may be carried out under reduced pressure or under elevated pressure.

The reaction can be carried in the air. However, it is carried out preferably in an inert gas atmosphere of e.g. nitrogen or argon. The reaction time is preferably at least 0.5 hour and at most 24 hours. A longer reaction tends to produce by-products such as a hydroxyphthalic acid and a substituted benzoic acid. The reaction is initiated usually by heating the reaction raw materials to a predetermined reaction temperature with properly stirring. After completion of the reaction, in accordance with a known method, the reaction mixture is subjected to hot filtration to remove insoluble components, and then the reaction mixture is cooled, whereby a crude ODPA will be deposited and recovered.

In a case where the reaction mixture has a high viscosity when subjected to hot filtration, it may be diluted with a solvent used in the reaction and then subjected to hot filtration.

In addition to the crude ODPA obtained by any one of the above-described processes (1-1) to 1-3), ODPA synthesized by an oxidation reaction of tetramethyldiphenyl ether as described in JP-B-7-107022 may also be used to obtain a high purity ODPA by the present purification process.

Further, a substance having a part of or the entire ODPA hydrolyzed may also be used. The hydrolyzate of ODPA may be converted to an anhydride by dehydration at a temperature in the vicinity of the temperature in a vacuum heat treatment step as described hereinafter, as already described for conversion of the oxydiphthalic acid to an anhydride in (1-2). However, since a part of the hydrolyzate is decomposed by decarboxylation reaction in the vacuum heat treatment step thereby to decrease the polymerizability of the ODPA, the content of the hydrolyzate in the ODPA immediately before the vacuum heat treatment step is desirably usually at most 50% preferably at most 15% more preferably at most 5%, as calculated as a semi-hydrolyzate having one of the two acid anhydride groups in the ODPA hydrolyzed.

### (1-4) Crude ODPA

The crude ODPA thus obtained contains impurities derived from the production process. The crude ODPA obtained in the above-described (1-1) and (1-2) contain impurities containing mainly nitrogen atoms. Namely, the nitrophthalic acid, its anhydride or phthalimide as the reaction raw material remains. Further, due to insufficient hydrolysis of the imide a substance having one of the two acid anhydride groups in ODPA being an imide group, and further, the reaction solvent such as N,N-dimethylacetamide, and a substance derived from the nitrous acid added at the time of reaction, are contained. The amount of such substances contained in the ODPA varies depending upon the production process, and the nitrogen atom content is usually at least 14 μmol/g and at most 100 μmol/g.

On the other hand, the crude ODPA obtained in (1-3) contains impurities containing mainly a halogen atom and a phosphorus atom. For example, an unreacted halogenated phthalic anhydride raw material, a high boiling point halogen-containing reaction solvent such as orthodichlorobenzene or a trichlorobenzene, a tetraphosphonium salt as a phase-transfer catalyst or an ionic substance added as a promoter, and further, unidentified reaction by-products or coloring substances are contained as impurities. Particularly the tetraphenylphosphonium salt is hardly soluble in water or an organic solvent and is hardly sublimated, whereby it will hardly be removed. The content of each of these substances varies depending upon the production process, and the total halogen atom content is usually at most 10 μmol/g and at most 500 μmol/g, and the phosphorus atom content is usually at least 1 μmol/g and at most 500 μmol/g.

Further, as an impurity in common with all the crude ODPA, fine insoluble particles which are impurities irrespective of the production process are contained. The fine insoluble particles mean impurities which are not soluble at room temperature in a solvent to be used at the time of polymerization for the polyimide, such as N,N-dimethylacetamide or N-methylpyrrolidone. Such impurities include, in addition to impurities which are originally contained in the raw material, impurities mixed in the production process and impurities mixed in a process handling the ODPA after the production. The former may, for example, be a catalyst powder, a metal powder and ones derived from a production apparatus such as a packing powder, and the latter may, for example, be a fine powder such as a dust floating in the atmosphere in which the product is handled.

The content of the fine insoluble particles contained in the crude ODPA depends on the size of the fine insoluble particles. However, fine insoluble particles having a projected area diameter of from 5 to 20 μm are contained in an amount of usually at least 1,500 particles, preferably at least 2,000 particles, more preferably at least 3,000 particles, furthermore preferably at least 5,000 particles, particularly preferably at least 10,000 particles, per 1 g of the crude ODPA.

Further, fine insoluble particles having a projected area diameter of at least 20 μm are contained in an amount of usually at least 250 particles preferably at least 500 particles, more preferably at least 1,000 particles, particularly preferably at least 1500 particles, per 1 g of the crude ODPA.

Further, the ODPA has three types of isomers including the 3,3′-form, the 3,4′-form and the 4,4′-form by the difference of the position of the ether bond. The position of the ether bond corresponds to the position of the substituent on the substituted phthalic acid as the raw material for production of the ODPA. The crude ODPA in the present application may be either a composition consisting of a single isomer or a mixture of a plurality of isomers.

### (2) Purification of Crude ODPA

The crude ODPA is purified by a procedure comprising the step A and the step B.

In the present specification a high purity ODPA means an ODPA subjected to both the purification procedure of the step A and the step B, and the crude ODPA means an ODPA not subjected to the steps A and B, or subjected to only one of the steps A and B.

(2-1) Step A: A Step of Heating the Crude ODPA to a Temperature of at Least 150° C. and at Most 350° C. to Evaporate and/or Sublimate it, and Condensing and Recovering the Evaporated and/or Sublimated Vapor

(a) Crude ODPA to be Used in this Step

The crude ODPA to be subjected to this process is not particularly limited, and the nitrogen content of the crude ODPA is usually at most 14 μmol/g, preferably at most 10 μmol/g, more preferably at most 1 μmol/g, furthermore preferably at most 0.1 μmol/g. If the nitrogen content is high, the ODPA to be obtained in this step tends to have a deteriorated color tone and be colored red. The crude ODPA produced by the above production process (1-2) tends to contain a nitrogen-containing compound. The phosphorus content of the crude ODPA is usually at most 50 μmol/g, preferably at most 10 μmol/g, more preferably at most 1 μmol/g, furthermore preferably at most 0.5 μmol/g, most preferably at most 0.1 μmol/g. If the phosphorus content is high, decomposition of the ODPA tends to be accelerated. The crude ODPA produced by the above production process (1-3) tends to contain a phosphorus-containing compound.

On the other hand, in this step, the fine insoluble particles, phosphorus and halogen can be removed. When the crude ODPA to be subjected to this step contains fine insoluble particles in an amount of usually at least 1,500 particles, preferably at least 2,000 particles, more preferably at least 3,000 particles, furthermore preferably at least 5,000 particles, particularly preferably at least 10,000 particles the fine insoluble particles can be efficiently removed. Further, as mentioned above, the phosphorus content is preferably low so as to suppress the decomposition of the ODPA. However, from a crude ODPA containing phosphorus in an amount of 10 μmol/g or more, such phosphorus can efficiently be removed. Further, from a crude ODPA containing halogen in an amount of 0 μmol/g or more, halogen can efficiently be removed.

(b) Evaporation and/or Sublimation of Crude ODPA

The evaporation and/or sublimation is carried out at a temperature of at least 150° C. and at most 350° C. It is carried out at a temperature of preferably at least 170° C., more preferably at least 200° C., furthermore preferably at least 228° C. Further, it is carried out at a temperature of preferably at most 330° C., more preferably at most 310° C., furthermore preferably at most 299° C. If the temperature is low, evaporation and/or sublimation of the ODPA will not efficiently be carried out. On the other hand, if the temperature is high, the ODPA is likely to be decomposed or colored.

The pressure is not particularly limited, but it is carried out usually under reduced pressure. Specifically, it is carried out under a pressure of usually at most 4,000 Pa, preferably at most 3,000 Pa, more preferably at most 2,000 Pa. When the pressure is lowered, evaporation and/or sublimation of the ODPA will efficiently be carried out.

The oxygen concentration in the vapor phase portion in the system in which the evaporation and/or sublimation is carried out, is preferably as low as possible. Specifically, it is usually at most 500 ppm, preferably at most 100 ppm, more preferably at most 50 ppm, furthermore preferably at most 10 ppm. If the oxygen concentration in the system is high, the ODPA is likely to be decomposed or colored.

If the evaporation rate is too high, the fine insoluble particles and other impurities will not sufficiently be removed due to the entrainment, and the too low evaporation rate is unfavorable from the economical viewpoint. Accordingly, the proper evaporation and/or sublimation rate should be selected by controlling the temperature and the pressure. As the evaporation and/or sublimation rate, the linear velocity of the vapor is usually at most 4 m/sec, preferably at most 2 m/sec, more preferably at most 1.5 m/sec, particularly preferably at most 1 m/sec.

Whether the ODPA is sublimated from the solid or evaporated from the melt depends on the isomers of the ODPA to be used and the impurities contained. For example, since 4,4′-ODPA has a melting point of about 228° C., it is sublimated from the solid when the heating temperature is lower than the melting point, and it is evaporated from the melt when the heating temperature is higher than the melting point.

(c) Recovery of Evaporated and/or Sublimated ODPA

Then, the evaporated and/or sublimated ODPA is cooled to an appropriate temperature, whereby the ODPA vapor is recondensed and recovered. The cooling temperature for the ODPA vapor is usually at most 150° C., preferably at most 100° C., more preferably at most 50° C. As the cooling method, various known methods may be employed. However, usually the vapor is deposited, solidified and recovered in a condenser installed at an appropriate space in the vapor phase in the apparatus for evaporating and/or sublimating the ODPA by vacuum heating. A plate-shape condenser is preferred. The ODPA deposited and solidified on the plate-shape condenser will easily be scraped and recovered with an appropriate scraping apparatus.

(d) ODPA after Step A

By this step, the content of particularly the fine insoluble particles in the ODPA can be reduced. Namely, the content of the fine insoluble particles in the ODPA purified by this step can be reduced to at most one fifth, preferably at most one tenth, more preferably at most one twenties, of the content before the purification. Specifically, the content of the fine insoluble particles having a projected area diameter of from 5 to 20 μm per 1 g of the ODPA can be reduced to usually at most 3,000 particles, preferably at most 2,000 particles, more preferably at most 1,500 particles, furthermore preferably at most 1200 particles.

Further, by this step, the phosphorus content can be reduced. In a case where the ODPA to be subjected to this step contains phosphorus, the phosphorus content in the ODPA purified by this step can be reduced to at most one tenth, preferably at most one hundredth, more preferably at most one two-hundredth, of the content of the ODPA before the purification. Specifically, it can be reduced to at most 40 μmol/g, preferably at most 10 μmol/g, more preferably at most 1 μmol/g, furthermore preferably at most 0.1 μmol/g, particularly preferably at most 0.1 μmol/g.

Further, by this step, the halogen content can be reduced. In a case where the ODPA to be subjected to this step contains halogen, the halogen content in the ODPA purified by this step can be reduced to at most half, preferably at most one fifth, more preferably at most one tenth, of the content of the ODPA before the purification. Specifically the content can be reduced to at most 9 μmol/g, preferably at most 8.5 μmol/g, more preferably at most 5 μmol/g, furthermore preferably at most 1 μmol/g.

Further, it is preferred to adjust the conditions of this step so that the contents of the fine insoluble particles, phosphorus and halogen in the ODPA purified by this step will be within the above ranges. Accordingly, the evaporation and/or sublimation rate is adjusted so as to lower the linear velocity of the vapor. The linear velocity of the vapor will be lowered by decreasing the temperature or increasing the pressure for the evaporation and/or sublimation.

(2-2) Step B: Step of Washing the Crude Oxydiphthalic Anhydride with at Least One Solvent Selected from an Organic Acid Having at Most 6 Carbon Atoms, and an Organic Ester or a Ketone Having at Most 12 Carbon Atoms in an Amount of from 0.5 to 20 Times by Weight to the Crude Oxydiphthalic Anhydride

In this washing step, the crude ODPA is washed with an organic solvent. Usually, an ODPA solution or slurry is stirred.

(a) Crude ODPA to be Used in this Step

The crude ODPA to be subjected to this step is not particularly limited. However, since particularly a nitrogen atom-containing compound in the ODPA can be removed in this step, a crude ODPA having a nitrogen content of usually at least 0.5 μmol/g, preferably at least; 1 μmol/g, more preferably at least 10 μmol/g furthermore preferably at least 14 μmol/g, can be suitably used.

(b) Solvent

The organic solvent is not particularly limited, and the boiling point under normal pressure is usually at most 250° C., preferably at most 200° C., more preferably at most 150° C., and usually at least 0° C., preferably at least 10° C., more preferably at least 30° C., furthermore preferably at least 50° C.

Specifically, an aromatic compound such as toluene, benzene, xylene or chlorobenzene; an organic acid having at most 6 carbon atoms such as acetic acid, formic acid or propionic acid; a ketone such as acetone, methyl ethyl ketone, diethyl ketone or methyl isobutyl ketone; or an organic ester having at most 12 carbon atoms such as methyl acetate or butyl acetate, is preferably used.

Among them an organic acid having at most 6 carbon atoms, an organic ester having at most 12 carbon atoms and/or a ketone having at most 12 carbon atoms, is preferred. Ethyl acetate and/or acetic acid is more preferred.

Such organic solvents may be used alone or as mixed.

(c) Washing Conditions

All the ODPA may be dissolved in a solvent, or washing may be carried out in the form of a suspension.

As the amount of the solvent to be used based on the weight of the ODPA raw material the lower limit is usually at least 0.5 time, preferably at least one time, and the upper limit is usually at most 20 times, preferably at most 10 times.

The washing temperature is not particularly limited so long as the solvent is in a liquid phase. It is usually at least 0° C., preferably at least 25° C., more preferably at least 50° C., and usually at most the boiling point of the solvent, preferably at most 250° C., more preferably at most 200° C., furthermore preferably at most 150° C. The temperature is preferably higher so as to increase the washing efficiency.

Although the pressure is not particularly limited, washing is carried out under a pressure of at least the atmospheric pressure so as to increase the washing efficiency. It is possible to carry out washing at a temperature of at least the boiling point of the solvent by means of a reactor capable of elevating the pressure.

The time for washing is usually at least 1 minute, preferably at least 10 minutes, more preferably at least 30 minutes and usually at most 12 hours, preferably at most 6 hours, more preferably at most 3 hours.

After the washing, the temperature of the solvent is brought to be room temperature, and then the solid ODPA is collected by filtration with e.g. a filter paper to recover the ODPA.

(d) ODPA after Step B

By this step, particularly the nitrogen content in the ODPA can be reduced. Namely, the nitrogen content of the ODPA purified by this step is usually at most 14 μmol/g, preferably at most 10 μmol/g, more preferably at most 1 μmol/g, furthermore preferably at most 0.5 μmol/g

### (2-3) Order of Steps A and B

The order of such purification steps is not particularly limited, but the preferred order varies depending upon the process for producing the crude ODPA.

When the crude ODPA obtained by the production process (1-2) is used, the crude ODPA usually contains nitrogen-containing organic impurities, and the nitrogen-containing organic impurities usually have a high solubility in the washing liquid as compared with the ODPA. Accordingly, it is more preferred to carry out the washing step before the vacuum heat treatment.

Namely, the crude oxydiphthalic anhydride produced by the process employing the substituted phthalimide as the raw material is preferably subjected to the step B: a step of washing it with at least one solvent selected from an organic acid having at most 6 carbon atoms, and an organic ester or a ketone having at most 12 carbon atoms in an amount of from 0.5 to 20 times the weight of the crude oxydiphthalic anhydride, and then subjected to the step A: a step of heating the crude oxydiphthalic anhydride to a temperature of at least 150° C. and at most 350° C. to evaporate and/or sublimate it, and condensing and recovering the evaporated and/or sublimated vapor.

On the other hand, when the crude ODPA obtained by the production process (1-3) is employed, the crude ODPA usually contains a phosphorus-containing compound, and even when it is washed, no sufficient effect may be obtained by influences of e.g. the remaining phase-transfer catalyst component. Accordingly, it is preferred to carry out the vacuum heat treatment to remove such a compound and then carry out the washing step.

Namely, the crude oxydiphthalic anhydride produced by the process employing a phthalic acid substituted by a halogen atom as the raw material, is preferably subjected to the step A: a step of heating the crude oxydiphthalic anhydride to a temperature of at least 150° C. and at most 350° C. to evaporate and/or sublimate it, and then condensing and recovering the evaporated and/or sublimated vapor, and then subjected to the step B: a step of washing the crude oxydiphthalic anhydride with at least one solvent selected from an organic acid having at most 6 carbon atoms, and an organic ester or a ketone having at most 12 carbon atoms in an amount of from 0.5 to 20 times the weight of the crude oxydiphthalic anhydride.

### (2-4) Other Steps

A known purification step such as recrystallization, pulverization or drying may further be additionally carried out before or after the combination of the steps A and B or between the steps A and B. However, in a case where the additional purification step is carried out after the combination of the steps A and B, in order to reduce inclusion of fine insoluble particles present in the production environment as far as possible, it is preferred to keep the cleanness of the working environment of at most class 4 as defined by JIS B9920.

Recrystallization is carried out usually from a high boiling point solvent such as dimethyl sulfoxide, sulfolane, N,N-dimethylformamide, N,N-dimethylacetamide, N-methylpyrrolidone, hexamethylphosphoric triamide or a dichlorobenzene, a dichlorotoluene or a trichlorobenzene. As the amount of the solvent to be used, the minimum amount required is such an amount that the ODPA can be completely dissolved at the boiling point of the solvent under the atmospheric pressure, and preferably the amount is at least 1 time and at most 20 times the weight of the ODPA. Under elevated pressure, the washing may be carried out at a temperature higher than the boiling point of the solvent. However, the ODPA will be decomposed when the temperature is too high. Accordingly, the temperature at the time of dissolution is usually at most 250° C., preferably at most 200° C. After dissolution, the solution is cooled to a temperature of at most room temperature, whereupon deposited solid is collected by filtration to recover the ODPA.

Pulverization is carried out, when the particle size of the crude ODPA is relatively large, for the purpose of improving the washing efficiency. The pulverization may be carried out by means of a ball mill, a jet mill or another pulverizer, and is carried out preferably in an inert gas atmosphere of e.g. nitrogen containing no water, so as to prevent coloring or hydrolysis of the ODPA by heat generation at the time of the pulverization. The pulverization is carried out so that the particle size of the ODPA after the pulverization is usually at most 5 mm, preferably at most 1 mm, more preferably at most 500 μm furthermore preferably at most 100 μm, particularly preferably at most 50 μm.

Drying is carried out to remove the remaining solvent. It is carried out by heating the crude ODPA to from 50 to 150° C. It is carried out preferably under a pressure of at most the atmospheric pressure. The drying is carried out preferably in an inert gas atmosphere of e.g. nitrogen containing no water, so as to prevent coloring or hydrolysis of the ODPA by oxidation.

### 2. High Purity ODPA

By the above treatment, a high purity ODPA having a content of fine insoluble particles having a projected area diameter of from 5 to 20 μm of at most 3,000 particles per 1 g. and having a light transmittance at 400 nm of at least 98.5% in a light path length of 1 cm when dissolved in acetonitrile at 4 g/L. Further, it is possible to bring the total content of halogen atoms to be at most 9 μmol/g, the nitrogen atom content to be at most 14 μmol/g and/or the phosphorus atom content to be at most 40 μmol/g.

### (1) Fine Insoluble Particles

The content of fine insoluble particles having a projected area diameter of from 5 to 20 μm is at most 3,000 particles preferably at most 2,000 particles more preferably at most 1,500 particles furthermore preferably at most 1,000 particles, per 1 g.

Further, the content of fine insoluble particles having a projected area diameter of larger than 20 μm, is usually at most 300 particles, preferably at most 200 particles, more preferably at most 100 particles, furthermore preferably at most 50 particles, per 1 g.

The content of the fine insoluble particles is determined in such a manner that the ODPA is dissolved in N-methylpyrrolidone, the solution is subjected to filtration through a filter, and the number of fine insoluble particles remaining on the filter paper is counted. The particle sizes and the number of the fine insoluble particles are measured by means of a microscopic method of measuring the sizes and the number of the fine insoluble particles on a microscopic image. Specifically, they can easily be measured by a particle size image processor such as XV-1000 manufactured by KEYENCE CORPORATION. In the present invention, the projected area diameter which is a diameter of a circle having the same area as the projected area of the fine insoluble particle, and which is also called the Heywood diameter, is employed.

A polyimide is used mainly as a film or a surface protective membrane of a semiconductor. In such a case, when a large amount of fine insoluble particles having a projected area diameter of from 5 to 20 μm, which have about the same sizes as the thickness of the film, are contained, specifically, when they are contained in an amount of at least 3,000 particles per 1 g of the ODPA, mechanical strength of the film or the like will be influenced in order that the influence is sufficiently suppressed, the content of the fine insoluble particles is required to be low. The content of the fine insoluble particles having sizes larger than 20 μm is low as compared with that of the fine insoluble particles having sizes of from 5 to 20 μm, and further, the fine insoluble particles having sizes smaller than 5 μm are usually small as compared with the thickness of the polyimide film or the polyimide membrane. Accordingly, the influences of the fine insoluble particles having these sizes over the quality of ODPA are relatively small as compared with those of the fine insoluble particles of from 5 to 20 μm.

### (2) Light Transmittance

The solution having the high purity ODPA dissolved in acetonitrile at 4 g/L, has a light transmittance at 400 nm in a light path length of 1 cm of at least 98.5%, preferably at least 98.7%, more preferably at least 99.0%.

The transmittance of the high purity ODPA is measured at room temperature under normal pressure by means of an UV/visible absorptiometer over wavelengths of from 800 to 200 nm with respect to a sample having the ODPA dissolved in acetonitrile at 4 g/L in a quartz cell with a light path length of 1 cm. The transmittance of the ODPA relates to the content of impurities. The coloring impurities cause a significant decrease of the transmittance in the vicinity of 400 nm, inhibit polymerization of ODPA with a diamine, decrease the strength of the polyimide film and cause deterioration of the color tone of the film.

The transmittance is measured in such a manner that 100 mg of the ODPA is dissolved in acetonitrile (for liquid chromatography, manufactured by KANTO CHEMICAL CO., INC.) at room temperature and the volume of the solution is brought to be 25 ml, the solution is put in a quartz cell with a light path length of 1 cm, and the absorbance is measured by means of an UV/visible spectrophotometer (UV-1600PC manufactured by Shimadzu Corporation). The measurement range is from 200 to 800 nm, and the resolution is at most 0.5 nm. In a case where the dissolution rate of ODPA crystals in acetonitrile is low, the ODPA crystals may be dissolved while applying ultrasonic waves by means of a commercial ultrasonic cleaner.

### (3) Nitrogen Impurities

The nitrogen atom content is usually at most 14 μmol/g, preferably at most 13 μmol/g, more preferably at most 12 μmol/g.

The nitrogen atom content is quantitatively analyzed in accordance with a known method by a chemoluminescence method after oxygen combustion. On that occasion the detection limit has to be set to be at most 3 ppm.

The impurities containing nitrogen atoms are present mainly in the form of an imide or a nitrophthalic acid, and not only inhibit polymerizability to impair physical properties of the polyimide but may cause coloring.

### (4) Halogen Impurities

The halogen atom content is usually at most 9 μmol/g, preferably at most 8.5 μmol/g, more preferably at most 5 μmol/g, furthermore preferably at most 1 μmol/g.

Fluorine, chlorine and bromine are quantitatively analyzed in accordance with a known method by subjecting the ODPA to oxygen combustion and letting the sample be absorbed in an aqueous hydrogen peroxide/alkali solution, followed by quantitative analysis by a calibration method by means of ion chromatography.

Iodine is quantitatively analyzed in accordance with a known method by subjecting the ODPA to oxygen combustion and letting the sample be absorbed in an aqueous hydrazine solution, followed by quantitative analysis by a calibration method by means of ion chromatography.

### (5) Phosphorus Impurities

The phosphorus atom content is usually at most 40 μmol/g, preferably at most 10 μmol/g, more preferably at most 1 μmol/g, furthermore preferably at most 0.5 μmol/g, particularly preferably at most 0.1 μmol/g.

The phosphorus content is determined in accordance with a known method by means of ICP-AES employing a sample after wet decomposition. The detection limit has to be set to be at most 3 ppm.

### 3. Polyimide

### Method for Producing Polyimide

The high purity ODPA of the present invention may be reacted with a diamine to obtain a polyimide containing oxydiphthalic anhydride units and diamine units by a known method. Namely, the high purity ODPA and a diamine are mixed in a solvent to obtain a polyamic acid, and the polyamic acid is heated to obtain a polyimide.

The diamine to be used is not particularly limited, and it is suitably selected depending upon the application from various aromatic diamines and alicyclic diamines. Particularly, for application as a surface protective membrane and a transparent polyimide film as semiconductor materials, preferred is an aromatic diamine which has both relatively low molecular weight and heat resistance, and with which thickness and the degree of polymerization tend to be increased. Among them, a phenylenediamine, a toluenediamine, a methylenedianiline, an oxydianiline, a thiodianiline, a sulfonyldianiline, a benzophenonediamine, a tolidine, etc may be used, an oxydianiline, a sulfonyldianiline or a benzophenonediamine is preferred, and 4,4′-oxydianiline is most preferred. As such a diamine, one sufficiently purified by a known method is used.

When the high purity ODPA of the present invention is used as a dicarboxylic acid components a polyamic acid which has a sufficiently high viscosity as a polyamic acid and which is less colored can be produced. Namely, such an excellent polyamic acid can be obtained that the polyamic acid with 4,4′-oxydianiline in a N,N-dimethylacetamide solvent having a polymer concentration of 15 wt %, has an inherent viscosity of at least 1.6 dL/g, preferably at least 1.8 dL/g, more preferably at least 2.0 dL/g, and a transmittance at 400 nm of at least 55%.

### (2) Physical Properties of Polyimide of the Present Invention

By use of the high purity ODPA, a tough polyimide film having high strength can be obtained. Namely, the polyimide containing oxydiphthalic anhydride units and diamine units of the present invention has a breaking extension of at least 20% preferably at least 25%. Further, it has a breaking stress of at least 130 MPa, preferably at least 150 MPa.

The breaking extension and the breaking stress in the present invention are averages of six measurements with respect to a polyimide film having a thickness of 20 μm, a length of 50 mm and a width of 10 mm in accordance with JIS K7113 under conditions of a temperature of 23° C. and a humidity of 55% with a distance between cramps (length at the stretched portion) of 20 mm at a tensile speed of 10 mm/min.

### 4. Application

The polyimide is usually applicable to a highly heat resistant plastic film having a glass transition temperature of at least 300° C., and is widely applicable to a flexible printed board represented by Kapton (tradename of DuPont) and UPILEX (tradename of UBE INDUSTRIES, LTD) and TAB (tape automated bonding). Such a film is required to have heat resistance and dimensional stability but is not required to be colorless and is usually colored orange to yellow. The acid anhydride which meets such requirements is pyromellitic anhydride or biphenyl tetracarboxylic anhydride. On the other hand, another application of the polyimide is a photosensitive polyimide. By imparting photosensitivity to a polyamic acid as the precursor of the polyimide, the polyimide is applicable also to a surface protective membrane of a semiconductor. In such a case, in addition to a sufficient heat resistance high transparency of the polyamic acid is required so as to prevent defective photosensitivity, and small amounts of ionic substances and fine insoluble particles in the raw material are required so as to prevent defects in semiconductor products.

The polyimide employing the high purity ODPA of the present invention has sufficiently high heat resistance, and is excellent in transparency and contains a small amount of impurities as compared with a conventional polyimide. Accordingly, it is suitable particularly as a raw material for a photosensitive polyimide applicable to a semiconductor.

### EXAMPLES

Now, the present invention will be explained in further detail with reference to Examples. However, the present invention is by no means restricted to the following Examples within a range not to exceed the scope of the present invention.

### Method of Measuring Transmittance

100 mg of a crude or purified ODPA was dissolved in acetonitrile (for liquid chromatography, manufactured by KANTO CHEMICAL CO., INC.) at room temperature and the volume of the solution was brought to be 25 ml, the solution was put in a quartz cell with a light path length of 1 cm, and the absorbance was measured by means of a UV/visible spectrophotometer (UV-1600PC, manufactured by Shimadzu Corporation). The measurement range was from 200 to 800 nm, and the resolution was at most 0.5 nm. In a case where the dissolution rate of the ODPA crystals in acetonitrile is low the ODPA crystals may be dissolved while applying ultrasonic waves by means of a commercial ultrasonic cleaner.

### Method of Measuring Nitrogen and Phosphorus Contents

The nitrogen content was quantitatively analyzed in accordance with a conventional method by means of a chemoluminescence method after oxygen combustion (TN-10 manufactured by DIA INSTRUMENTS CO., LTD.).

The phosphorus content was quantitatively analyzed in accordance with a known means. A sample was decomposed by a wet decomposition method employing a Kjeldahl flask to obtain a measurement solution. Quantitative analysis was carried out by a calibration method employing an inductively coupled plasma atomic emission spectrometer (JY38S manufactured by Jovin Yvon).

### Method of Measuring Halogen Element Contents

The total fluorine, the total chlorine and the total bromine were quantitatively analyzed in accordance with a known method by subjecting the ODPA to oxygen combustion, and letting the sample be absorbed in an aqueous hydrogen peroxide/alkali solution, followed by quantitative analysis by a calibration method by means of ion chromatography (DX500 manufactured by Dionex Corporation).

The total iodine was also quantitatively analyzed in accordance with a conventional method by subjecting the ODPA to oxygen combustion and letting the sample be absorbed in an aqueous hydrazine solution followed by quantitative analysis by a calibration method by means of ion chromatography (DX500 manufactured by Dionex Corporation).

### Method of Counting Fine Insoluble Particles

The fine insoluble particles contained in the ODPA were counted as follows.

### (1) Preparation of Solvent

Reagent grade N-methylpyrrolidone was passed through a filter with a mesh size of 0.2 μm in a clean bench of class 100 to remove fine insoluble particles with sizes of 0.2 μm or larger.

### (2) Preparation of Sample

In a clean bench of class 100, 1 g of a sample was accurately weighed in a washed and dried glass bottle, 200 ml of the above N-methylpyrrolidone was added and the mixture was subjected to an ultrasonic cleaner to dissolve the sample. With respect to the count of the ODPA in Comparative Examples as described hereinafter and ODPA raw materials 1 and 2 the samples had a high content of fine insoluble particles, and thus the samples were further diluted 100 times. Then, the solution thus obtained was passed through a filter with a mesh size of 0.45 μm to remove the fine insoluble particles by filtration

### (3) Count of Fine Insoluble Particles

In a clean room of class 1000 the number of fine insoluble particles on the filter was counted by means of a grain size image processor (XV-1000 manufactured by KEYENCE CORPORATION). The number of the counted fine insoluble particles was corrected by the sample weight and calculated as the number per 1 g of the sample.

### Crude ODPA 1

As the crude ODPA produced by the above process (I 2), 44′-ODPA: Lot 2004-11-03, manufactured by SUZHOU YINSHENG Chemical Co., Ltd.) was used. The results of analysis of the fine insoluble particle content, the nitrogen, phosphorus and halogen contents and the transmittance of the crude ODPA 1 are shown in Table 1

### Crude ODPA 2

As the crude ODPA produced by the above process (1-3), an ODPA produced by a process as described in the following Production Example 1 was used. The results of analysis of the fine insoluble particle content, the nitrogen, phosphorus and halogen contents and the transmittance of the crude ODPA 2 are shown in Table 2.

### Production Example 1

Produced in accordance with a process as described in Example 1 of Japanese Patent No. 3204641.

Namely, 150.37 g of 4-bromophthalic anhydride preliminarily purified by sublimation (Lot. AGN01, manufactured by TOKYO KASEI KOGYO CO., LTD.) and 250 g of orthodichlorobenzene (Lot. 707X2084, special grade, manufactured by KANTO CHEMICAL CO., INC.) were put in a 500 cc separable flask equipped with a mechanical stirrer and a reflux condenser, and they were stirred with heating in an oil bath until the internal temperature reached 195° C. in a nitrogen atmosphere. Then, 35.12 g of sodium carbonate (Lot. 707X1397, first grade, manufactured by KANTO CHEMICAL CO., INC.), 7.48 g of tetraphenylphosphonium bromide (Lot. FIG01, manufactured by TOKYO KASEI KOGYO CO., LTD.) and 3.48 g of potassium iodide (Lot. L37090E manufactured by KISHIDA CHEMICAL CO., LTD.) were dividedly charged in four times at 30-minutes intervals. After all the amount was charged, 100 g of 1,2,4-trichlorobenzene (Lot. EWN5441 manufactured by Wako Pure Chemical Industries Ltd.) was added, and reaction was carried out at an internal temperature of from 195 to 197° C. for 28 hours in total with stirring at about 300 rpm. Then, the reaction mixture was subjected to hot filtration with a Kiriyama funnel (SC-95W, No. 5B filter paper) equipped with an insulating jacket through which hot oil of 160° C. circulated, and the filtrate was cooled to room temperature. Deposited solid was subjected to filtration again, and the product collected by filtration was rinsed with 120 cc of toluene (special grade, manufactured by JUNSEI CHEMICAL CO., LTD.) at room temperature twice and then air-dried. 81.98 g of a pale red powder was recovered. The same reaction was repeated on the same scale to obtain 163.51 g in total of the crude ODPA. The results of analysis of the fine insoluble particle content, the nitrogen, phosphorus and halogen contents and the transmittance of the crude ODPA are shown in Table 2. With respect to the amount of the fine insoluble particles, coloring of the filter by the pre-treatment was significant, and the count by the grain size image processor was inhibited. The value in the Table represents the number of fine particles which could be counted.

### Production of High Purity ODPA Using Crude ODPA 1

### Example 1

### High Purity ODPA Purified by Carrying Out Step B and then Step A

165.0 g of the crude ODPA 1 and 500 cc of ethyl acetate (special grade, manufactured by JUNSEI CHEMICAL CO., LTD.) were put in a 1 L flask in a nitrogen atmosphere, and refluxed with heating for 2 hours in a state of a slurry. Then, the reaction mixture was cooled to about 15° C. and subjected to filtration, and the powder collected by filtration was washed with 80 cc of ethyl acetate once and dried to recover 160.29 g of a white powder.

35.07 g of this white powder and a magnetic stirrer made of Teflon were put in a 500 cc separable flask (manufactured by SHIBATA SCIENTIFIC TECHNOLOGY LTD., round bottom, band type) in a nitrogen atmosphere, and a cover for the separable flask, the inside of which can be air cooled and which is equipped with a collecting inner tube having a bottom with a diameter of 5 cm, was put. The separable flask was immersed in an oil bath of 265° C. for 85 minutes under a reduced pressure of from 70 to 60 Pa. 4,4′-ODPA in the reactor was melted and stirred. During this time, a nitrogen gas at room temperature was supplied to the collecting inner tube for cooling, while controlling the flow rate of the nitrogen gas so that the temperature of the discharged gas would not exceed 50° C. Then, the oil bath was removed, the separable flask was cooled to room temperature, and nitrogen was introduced to recover the pressure, to obtain a high purity 4,4′-ODPA attached to the collecting tube as white solid. The amount recovered was 31.65 g (90.2%). 2.00 g of gray solid remained at the bottom of the flask. The results of analysis of the fine insoluble particle content, the nitrogen, phosphorus and halogen contents and the transmittance of the recovered ODPA are shown in Table 1.

### Example 2

### High Purity ODPA Purified by Carrying Out Step A and then Step B

40.13 g of the crude ODPA 1 and a magnetic stirrer made of Teflon were put in a 500 cc separable flask (manufactured by SHIBATA SCIENTIFIC TECHNOLOGY LTD., round bottom, band type) in a nitrogen atmosphere, and a cover for the separable flask, the inside of which can be air cooled and which is equipped with a collecting inner tube having a bottom with a diameter of 5 cm, was put. The separable flask was immersed in an oil bath of 265° C. for 90 minutes under a reduced pressure of 40 Pa. 4,4′-ODPA in the reactor was melted and stirred. During this time, a nitrogen gas at room temperature was supplied to the collecting inner tube for cooling, while controlling the flow rate of the nitrogen gas so that the temperature of the discharged gas would not exceed 50° C. Then, the oil bath was removed, the separable flask was cooled to room temperature, and nitrogen was introduced to recover the pressure, to recover a high purity 4,4′-ODPA attached to the collecting tube as white solid 1.21 g of gray solid remained at the bottom of the flask.

The recovered ODPA was put in a 500 cc separable flask, and 120 g of ethyl acetate (special grade, manufactured by JUNSEI CHEMICAL CO., LTD.) preliminarily passed through a PTFE filter paper with a pore size of 0.5 μm was added, followed by stirring with heating under reflux for 1 hour in a nitrogen atmosphere. After the reaction mixture was cooled to room temperature, it was subjected to filtration in a clean box, and the recovered solid was dried under reduced pressure at room temperature for 2.5 hours. The yield was 36.90 g (92.0%). The results of analysis of the fine insoluble particle content, the nitrogen, phosphorus and halogen contents and the transmittance of the recovered ODPA are shown in Table 1.

### Comparative Example 1

### ODPA Purified Only by Step A

49.86 g of the crude ODPA 1 and a magnetic stirrer made of Teflon were put in a 500 cc separable flask (manufactured by SHIBATA SCIENTIFIC TECHNOLOGY LTD., round bottom, band type) in a nitrogen atmosphere, and a cover for the separable flask, the inside of which can be air cooled and which is equipped with a collecting inner tube having a bottom with a diameter of 5 cm, was put. The separable flask was immersed in an oil bath of 265° C. for 108 minutes under a reduced pressure of 50 Pa. 4,4′-ODPA in the reactor was melted and stirred. During this time, a nitrogen gas at room temperature was supplied to the collecting inner tube for cooling, while controlling the flow rate of the nitrogen gas so that the temperature of the discharge gas would not exceed 50° C. Then, the oil bath was removed, the separable flask was cooled to room temperature and nitrogen was introduced to recover the pressure, to recover a high purity 4,4′-ODPA attached to the collecting tube as white solid. The yield was 46.67 g (93.6%). 2.14 g of gray solid remained at the bottom of the flask. The fine insoluble particle content, the nitrogen phosphorus and halogen contents and the transmittance of the recovered ODPA are shown in Table 1.

### Comparative Example 2

### ODPA Purified Only by Step B

162.17 g of the crude ODPA 1 and 500 cc of ethyl acetate (special grade, manufactured by JUNSEI CHEMICAL CO., LTD.) were put in a 1 L flask in a nitrogen atmosphere and refluxed with heating for 2 hours in a state of a slurry. Then, the reaction mixture was cooled to about 15° C. and subjected to filtration, and the powder collected by filtration was washed with 80 cc of ethyl acetate once and dried to recover 157.66 g of a white powder. The results of analysis of the fine insoluble particle content, the nitrogen phosphorus and halogen contents and the transmittance of the recovered ODPA are shown in Table 1.

### Production of High Purity ODPA Using Crude ODPA 2

### Example 3

### High Purity ODPA Purified by Carrying Out Step B and then Step A

60.00 g of the crude ODPA 2 and 180 cc of ethyl acetate (special grade, manufactured by JUNSEI CHEMICAL CO., LTD.) and a magnetic stirrer made of Teflon were put in a 500 cc separable flask equipped with a reflux condenser in a nitrogen atmosphere. The separable flask was immersed in an oil bath, and the oil bath was heated to a temperature of 100° C., followed by reflux with heating for one hour at a stirring rate of about 200 rpm in a state of a slurry. Then, the reaction mixture was cooled to room temperature and subjected to filtration, and the powder collected by filtration was washed with 80 cc of ethyl acetate once and air dried to recover 57.05 g (95.1%) of a pale red powder. 30.04 g of this powder and a magnetic stirrer made of Teflon were put in a 500 cc separable flask (manufactured by SHIBATA SCIENTIFIC TECHNOLOGY LTD., round bottom, band type) in a nitrogen atmosphere, and a cover for the separable flask, the inside of which can be air cooled and which is equipped with a collecting inner tube having a bottom with a diameter of 5 cm was put. The separable flask was immersed in an oil bath of 265° C. for 2 hours under a reduced pressure of from 40 to 53 Pa. 4,4′-ODPA in the reactor was melted and stirred at a rate of 150 rpm. During this time, a nitrogen gas at room temperature was supplied to the collecting inner tube for cooling, while controlling the flow rate of the nitrogen gas so that the temperature of the discharged gas would not exceed 50° C. Then, the oil bath was removed, the separable flask was cooled to room temperature, and nitrogen was introduced to recover the pressure, to recover an ODPA attached to the collecting tube as white solid. The amount recovered was 24.58 g (81.8%). 4.3 g of black residue remained at the bottom of the flask. The results of analysis of the fine insoluble particle content, the nitrogen phosphorus and halogen contents and the transmittance of the recovered ODPA are shown in Table 2.

### Example 4

### High Purity ODPA Purified by Carrying Out Step A and then Step B

30.00 g of the crude ODPA 2 and a magnetic stirrer made of Teflon were put in a 500 cc separable flask (manufactured by SHIBATA SCIENTIFIC TECHNOLOGY LTD., round bottom band type) in a nitrogen atmosphere, and a cover for the separable flask, the inside of which can be air cooled and which is equipped with a collecting inner tube having a bottom with a diameter of 5 cm, was put. The separable flask was immersed in an oil bath of 265° C. for 120 minutes under a reduced pressure of 50 Pa. The ODPA in the reactor was melted and stirred at a rate of 150 rpm. During this time, a nitrogen gas at room temperature was passed through the collecting inner tube for cooling, while controlling the flow rate of the nitrogen gas so that the temperature of the discharged gas would not exceed 50° C. Then, the oil bath was removed, the separable flask was cooled to room temperature, and nitrogen was introduced to recover the pressure, to recover an ODPA attached to the collecting tube as white solid. The yield was 22.39 g (74.6%). Black residue remained at the bottom of the flask.

The recovered ODPA was pulverized and put in a 300 cc three-necked round bottom flask, and 60 cc of ethyl acetate (special grade, manufactured by JUNSEI CHEMICAL CO., LTD.) was added followed by stirring with heating under reflux for 1 hour in a nitrogen atmosphere. The reaction mixture was cooled to room temperature and subjected to filtration the product collected by filtration was rinsed about 50 cc of ethyl acetate, and the recovered solid was air dried at room temperature for 1 hour. The yield was 19.81 g (88.5%). The results of analysis of the fine insoluble particle content, the nitrogen, phosphorus and halogen contents and the transmittance of the recovered ODPA are shown in Table 2.

### Comparative Example 3

### ODPA Obtained Only by Step A

The ODPA in Example 3 subjected to the sublimation/recondensation step was taken out to obtain the ODPA of this Comparative Example. The results of analysis of the fine insoluble particle content, the nitrogen, phosphorus and halogen contents and the transmittance of the recovered ODPA are shown in Table 2.

### Comparative Example 4

### ODPA Purified Only by Step B

30.00 g of the crude ODPA 2 and a magnetic stirrer made of Teflon were put in a 500 cc separable flask (manufactured by SHIBATA SCIENTIFIC TECHNOLOGY LTD., round bottom, band type) in a nitrogen atmosphere, and a cover for the separable flask, the inside of which can be air cooled and which is equipped with a collecting inner tube having a bottom with a diameter of 5 cm, was put. The separable flask was immersed in an oil bath of 265° C. for 120 minutes under a reduced pressure of 50 Pa. The ODPA in the reactor was melted and stirred at a rate of 150 rpm. During this time, a nitrogen gas at room temperature was supplied to the collecting inner tube for cooling while controlling the flow rate of the nitrogen gas so that the temperature of the discharged gas would not exceed 50° C. Then, the oil bath was removed, the separable flask was cooled to room temperature, and nitrogen was introduced to recover the pressure, to recover an ODPA attached to the collecting tube as white solid. The yield was 23.64 g (78.8%). Black residue remained at the bottom of the flask. The results of analysis of the fine insoluble particle content, the nitrogen, phosphorus and halogen contents and the transmittance of the recovered ODPA are shown in Table 2. With respect to the amount of the fine insoluble particles, coloring of the filter by the pre-treatment was significant, and the count by the grain size image processor was inhibited. The value in the Table represents the number of fine particles which could be counted

### Evaluation of Polyimide Film

In order to show the effects of the production process of the present invention and the high purity ODPA of the present invention obtained by the process as a raw material for a polyimide film, polyimide films were prepared from the ODPA in Examples 1, 2 and Comparative Examples 1, 2 and the crude ODPA 1 in the same process, and their strength was evaluated.

Now, the process for producing a polyimide will be shown below.

In a nitrogen atmosphere, into a 500 cc reactor maintained at 25° C. by a circulating water, 3.638 g of 4,4′-oxydianiline (0.0182 mol, manufactured by Wakayama Seika Kogyo K.K.) preliminarily purified by distilled water and 52.0 g of N,N-dimethylacetamide at a dehydrated grade (manufactured by Wako Pure Chemical Industries Ltd., polymer concentration: 15 wt %) were put and dissolved. Then, 5.633 g (0.0182 mol) of the high purity ODPA prepared in Example 1 as a powder was dividedly charged over a period of about 30 minutes. Then, stirring was carried out at 250 for 6 hours.

1.3063 g of a polyamic acid solution to be obtained was diluted and dissolved in N,N-dimethylacetamide at room temperature, and the volume was brought to be 25 cc to prepare a sample for viscosity measurement. The sample concentration C was adjusted to be from 0.7 to 0.8 g/dL. C of this sample was 0.791 g/dL. This sample was put in a Ubbellohde viscometer (manufactured by SHIBATA SCIENTIFIC TECHNOLOGY LTD., the range of kinetic viscosity to be measured: 2 to 10 cSt) put in a thermostatic water bath of 30° C. and left at rest for at least 10 minutes, and then the falling time T between marks was measured, whereupon it was 350 seconds. The falling time Ts of N,N-dimethylacetamide as a solvent was 90 seconds. The inherent viscosity was calculated from the following formula:

Inherent viscosity={ln(T/Ts)}/C

In a clean box of class 1000, the obtained polyamic acid solution was cast on a glass plate by a doctor knife (coating thickness: 254 μm, width: 50 mm) and dried at room temperature for at least 12 hours. The film was separated from the glass plate and fixed on an aluminum plate flame (thickness: 0.5 mm, outer dimension: 110 mm×70 mm, dimensions of opening portion: 70 mm×30 mm) with clips, and heated in an electric furnace, the inside of which was replaced with nitrogen, at 120° C. for one hour, at 250° C. for one hour and then at 320° C. for 5 minutes for heat imidization. After the film was cooled to room temperature, it was cut out from the opening portion of the plate frame. The film thickness was from 0.0019 to 0.0020 mm.

This film was left at rest in an environment of 23° C. and a humidity of 55% for at least 12 hours, and a test specimen was cut out with a width of 10 mm from the film. The breaking strength of the test specimen was measured by using a tensile strength tester (TENSILON model RTC-1210A, manufactured by ORIENTEC CO., LTD.) (load full scale: 100 N, test rate: 10 mm/min, length of stretched portion: 20 mm, temperature: 23° C., humidity: 55%). Test was carried out six times, and the average of the measured values was obtained. The transmittance and the inherent viscosity of the polyamic acid, and the average breaking extension and the average breaking stress of the polyimide film are shown in Table 1.

TABLE 1
Impurity contents of ODPA and evaluation of polyimide properties

| | | | | Comp. | Comp. | Crude |
|---|---|---|---|---|---|---|
| | | Ex. 1 | Ex. 2 | Ex. 1 | Ex. 2 | ODPA 1 |
| ODPA | Fine insoluble particles | | | | | |
| | 5 to 20 μm particles/g | 520 | 1020 | 470 | 24200 | 25000 |
| | Longer than 20 μm particles/g | 40 | 80 | 50 | 1000 | 2000 |
| | Nitrogen atom content (μmol/g) | 7.86 | 11.43 | 14.29 | 7.14 | 15.00 |
| | Phosphorus atom content | <0.10 | <0.10 | <0.10 | <0.10 | <0.16 |
| | (μmol/g) | | | | | |
| | Halogen atom content (μmol/g) | | | | | |
| | F | —(*1) | <0.05 | <0.05 | <0.05 | <0.26 |
| | Cl | <0.03 | <0.03 | <0.03 | <0.03 | <0.03 |
| | Br | <0.01 | 0.01 | <0.01 | <0.01 | <0.01 |
| | I | — | — | — | — | <0.01 |
| | Total | <0.04 | <0.09 | <0.09 | <0.09 | <0.31 |
| | Light transmittance % (400 nm) | 99.55 | 99.27 | 98.03 | 98.52 | 96.36 |
| Polyamic | Light transmittance % (400 nm) | 59.1 | 66.8 | 53.2 | 50.4 | 35.5 |
| acid | Polymer viscosity (dl/g) | 1.72 | 2.07 | 1.59 | 1.53 | 1.19 |
| Polyimide | Breaking extension (%) | 26.6 | 26.01 | 22.8 | 13.3 | 10.1 |
| | Breaking stress (MPa) | 171.3 | 150.9 | 152.9 | 141.6 | 122.5 |

*1 −: not measured

TABLE 2
Impurity content of ODPA

| | | | | Comp. | Comp. | Crude |
|---|---|---|---|---|---|---|
| | | Ex. 3 | Ex.4 | Ex. 3 | Ex. 4 | ODPA 2 |
| ODPA | Fine insoluble particles | | | | | |
| | 5 to 20 μm particles/g | 840 | 1080 | 520 | >3110 (*2) | <3350 |
| | Longer than 20 μm particles/g | 140 | 90 | 80 | 260 | 300 |
| | Nitrogen atom content (μmol/g) | <0.07 | <0.07 | <0.07 | 0.29 | 0.07 |
| | Phosphorus atom content | 0.16 | <0.10 | 0.16 | 48.71 | 49.68 |
| | (μmol/g) | | | | | |
| | Halogen atom content (μmol/g) | | | | | |
| | F | 0.11 | <0.05 | <0.05 | 0.05 | <0.05 |
| | Cl | 0.48 | 0.03 | 0.39 | 118.31 | 126.76 |
| | Br | 4.01 | 0.08 | 4.13 | 6.13 | 7.76 |
| | I | 3.86 | 0.05 | 4.89 | 41.77 | 38.61 |
| | Total | 8.46 | 0.21 | 9.46 | 166.26 | 173.18 |
| | Light transmittance % (400 nm) | 98.94 | 98.73 | 98.16 | 91.84 | 91.76 |

*2: number of particles counted by automatic counter

### INDUSTRIAL APPLICABILITY

The high purity oxydiphthalic anhydride of the present invention is suitable as a raw material for a highly heat resistant and highly transparent polyimide or a high definition photosensitive polyimide in a field of electronic material production and semiconductor production.

The entire disclosure of Japanese Patent Application No. 2004-353696 filed on Dec. 7, 2004 including specification, claims and summary is incorporated herein by reference in its entirety.

1-12. (canceled)

13. A high purity oxydiphthalic anhydride, which has a content of fine insoluble particles having a projected area diameter of from 5 to 20 μm, of at most 3000 particles per 1 g, and has a light transmittance at 400 of at least 98.5% in a light path length of 1 cm when dissolved in acetonitrile at 4 g/L.

14. The high purity oxydiphthalic anhydride according to claim 13, which has a halogen atom content of at most 9 μmol/g.

15. The high purity oxydiphthalic anhydride according to claim 13, which has a nitrogen atom content of at most 14 μmol/g.

16. The high purity oxydiphthalic anhydride according to claim 14, which has a nitrogen atom content of at most 14 μmol/g.

17. A process for producing a high purity oxydiphthalic anhydride, which comprises purifying a crude oxydiphthalic anhydride by a procedure comprising the following steps A and B:
step A: a step of heating the crude oxydiphthalic anhydride to a temperature of at least 150° C. and at most 350° C. to evaporate and/or sublimate it, and condensing and recovering the evaporated and/or sublimated vapor;
step B: a step of washing the crude oxydiphthalic anhydride with at least one solvent selected from an organic acid having at most 6 carbon atoms and an organic ester or a ketone having at most 12 carbon atoms in an amount of from 0.5 to 20 times the weight of the crude oxydiphthalic anhydride.

18. The process for producing a high purity oxydiphthalic anhydride according to claim 17, wherein a crude oxydiphthalic anhydride obtained by reacting a halogenated phthalic anhydride with a carbonate or a halogenated phthalate, is purified by a procedure of carrying out the step A and then the step B.

19. The process for producing a high purity oxydiphthalic anhydride according to claim 17, wherein a crude oxydiphthalic anhydride obtained by coupling a substituted phthalimide, is purified by a procedure of carrying out the step B and then the step A.

20. A process for producing a high purity oxydiphthalic anhydride, which comprises heating a crude oxydiphthalic anhydride having a nitrogen atom content of at most 14 μmol/g to a temperature of at least 150° C. and at most 350° C. to evaporate and/or sublimate it, and condensing and recovering the evaporated and/or sublimated vapor.

21. The process for producing a high purity oxydiphthalic anhydride according to claim 20, wherein the crude oxydiphthalic anhydride is a crude oxydiphthalic anhydride obtained by coupling a substituted phthalimide.

22. A high purity oxydiphthalic anhydride produced by the production process as defined in any one of claim 17.

23. A polyimide obtained by polymerizing the high purity oxydiphthalic anhydride as defined in any one of claim 13, with a diamine.

24. A polyimide obtained by polmerizing the high purity oxydiphthalic anhydride as defined in any one of claim 22 with a diamine.

25. A polyimide containing oxydiphthalic anhydride units and diamine units, wherein a film of the polyimide having a thickness of 20 μm, a length of 50 mm (a length at a stretched portion of 20 mm) and a width of 10 mm, has a breaking extension of at least 25% as measured in accordance with JIS K7113.

26. The polyimide containing oxydiphthalic anhydride units and diamine units according to claim 25, wherein a film of the polyimide having a thickness of 20 μm, a length of 50 mm (a length at a stretched portion of 20 mm) and a width of 10 mm, has a breaking stress of at least 130 MPa as measured in accordance with JIS K7113.
